(19) United States
(12) Patent Application Publication
(10) Pub. No.: US 2009/0131539 A1
(43) Pub. Date: May 21, 2009

(54) Mixture

(75) Inventor: Ralf Schutz, Mannheim (DE)

Correspondence Address: HUDAK, SHUNK & FARINE, CO., L.P.A., 2020 FRONT STREET, SUITE 307, CUYAHOGA FALLS, OH, 44221, US

(73) Assignees: Frank SCHNEIDER, Ludwigshafen (DE); Ralf SCHÜTZ, Mannheim (DE)

(21) Appl. No.: 11/988,617

(22) PCT Filed: Jul. 12, 2006
(86) PCT No.: PCT/EP2006/064164

§ 371 Date: Jan. 11, 2008

(30) Foreign Application Priority Data
Jul. 12, 2005 (DE) 10 2005 032 918.7

(51) Int. Cl. A61K 31/05; A61P 43/00; A01N 31/08; A01P 17/00

(52) U.S. Cl. 514/731

(57) ABSTRACT

A mixture of substances, in particular a biocide for combating human and animal parasites comprising a) an A component containing at least one type of alcohol of general formula R1-OH or R1-OH—R1′, wherein R1 and/or R1′ is an alkyl radical associated with a hydroxyl group which comprises from 1 to 12, preferably from 1 to 6, carbon atoms, b) a B component consisting of at least one type of carbonyl compound of formula R2-CO—R2′, wherein R2 and/or R2′ is an aryl-, alkyl- or cycloalkyl radical associated with a carbonyl group containing from 1 to 5, preferably from 1 to 2, carbon or hydrogen atoms, and c) at least one type of essential oil in the form of a C component consisting of an aromatic hydroxy compound of formula R3-0H, wherein R3 is a compound of at least one type of cyclic aryl radical which can be substituted up to two times and at least one organic compound R3′, wherein R3′ is an aryl-, alkyl- or cycloalkyl radical, wherein the components A and B are brought into reaction with hydrogen peroxide and the component C is subsequently mixed.

### FIELD OF THE INVENTION

The invention relates to a mixture, in particular a biocide or insecticide for topical or internal therapeutical treatment of invertebrates and vertebrates, particularly insects and arthropods in case of infestation with pest animals. In addition the invention relates to usage of such a mixture for producing an appropriate medicine for treatment of insects. Pests in this context are classified as belonging to the group of invertebrates.

### BACKGROUND OF THE INVENTION

Numerous mixtures or biocides are known already for controlling pests like e.g. Varroa mite which also infests honey bees and their larvae or Acarapis woodi mite (USA). These substances are not approved in several states and some of them are only available on prescription.

Especially in the treatment of beneficial insects like honeybees it is important that on the one hand as few bees as possible die of the treatment and on the other hand the biocide can be applied with the least possible amount of residues, i.e. that no residues are left in the bees product like honey or wax. In cases where residues of the biocide are unavoidable due to its concentration or application the biocide should not constitute a human health hazard.

Formic acid and lactic acid are two of the few known biocides or insecticides which can be applied without residues or at least without any damage to human health. Mortality rate among the beneficial insects, however, is still a problem in these cases.

The products available under the trade names of “Apiguard®” containing the active agent Thymol, “Bayvarol®” with the active agent Flumethrin and “Perizin®” with the active agent Coumaphos are biocides which are partially regarded as a problem for human health because of residues found in honey products.

In addition biocides are known under the trade names of “Apistan®”, “Apitol®”, Apivar®”, Supona®”, “Birlane®”, Asuntol®”, “TakTic®”, and “Bumentran®” none of which is approved in Germany because of health hazard related to residues in honey products and wax.

Numerous attempts to destroy mites, especially Varroa mite or round worms (nematodes) or at least to contain them on a long-term basis without applying any substances hazardous to human health have so far been unsuccessful. In 2003 the Varroa mite infesting bee colonies caused a damage to the tune of 10 billion US$ in the USA alone.

It was found that even highly effective biocides and insecticides lost in effectiveness in recent years as a result of growing resistance of the pests.

Control of varroatosis by means of those conventional, commercially available acaricides obviously does not bring about the desired results. It is known that alternatively various essential oils, i.e. volatile natural or nature identical as well as synthetic aromatic substances on the basis of an aromatic hydroxyl compound in varying concentrations can be used for pest control.

It is, however, a problem to dispense the substances in ways and amounts that do not harm the beneficial insects. When it comes to essential oils and chemically pure substances there is the problem of solubility in water. Aromatic hydroxyl compounds are mostly based on phenols or phenol esters which are almost insoluble in water just as phenol itself.

On a lab scale control of the amount dispensed can be guaranteed by a complicated test set-up. In practical in-the-field applications like in bee hives so far no satisfactory solution has been found for keeping the amount dispensed sufficiently high and yet below the harmful threshold.

In field tests pure essential oils were applied by way of evaporation into the ambient air or partly by direct application onto the pests. It was not possible by these methods so far to achieve a repeatable concentration which was at the same time fast and sufficiently effective and did not cause any serious damage among the beneficial insects.

From DE 600 14 100 T2 it is known—as specified in par. 0027—that ketones like acetone or alcohols can be used as carriers for pesticide mixtures. Two sample formulations are described in which acetone is used. In sample formulation under par. 0041 phenyl propionate is dissolved in acetone and mixed with rosemary oil, peppermint oil and eugenol. In sample formulation under par. 0043 methyl salicylate is dissolved in acetone and mixed with peppermint oil, eugenol, and phenethyl propionate.

In US 2003-0194454 page 18 et seq. use of between 5% up to 75% of one or several essential oils based on an appropriate carrier substance is described for pest control. To achieve a sufficiently high mortality rate of the pests by this method concentrations of 35% on the average are required. With this method there is a great risk, however, of essential oil in exceedingly high concentrations damaging the beneficial insect, i.e. not only the pests but also the patient, in the described cases the bees will die. If the concentration of e.g. cloves oil is too high more than 90% of the bees in this hive will die.

### SUMMARY OF THE INVENTION

The point is to provide a mixture and hence a form of application which allows a non-hazardous use of an essential oil, i.e. without any negative effect on the beneficial insect yet leading to a pest mortality rate of at least 95% and in the case of bees with hardly any residues in honey so that the mixture can be used without a human health hazard involved.

### DETAILED DESCRIPTION OF THE INVENTION

It was found out by coincidence that a mixture consisting of a primary and/or secondary alcohol as component A, a ketone as component B, and an essential oil substance as component C will be more effective by a factor of 300 as measured by the pest mortality rate compared with a mixture of water, essential oil and acetone, if an oxidising agent like hydrogen peroxide (H₂O₂) is added to the mixture as component D. The oxidising agent is preferably mixed with component A and B thus triggering a redox reaction through which the oxidising agent is reduced and at least one of the components A and B is partially oxidized. Immediately after that or after mixing in further components, component C is mixed in together with component D. This so-called oxide component OX arising from oxidation of components A and/or B brings about an improvement in the sense that almost all pests are destroyed while the beneficial insect remains unharmed.

Due to these substances acting together a synergistic effect is achieved: Essential oil concentration is reduced from an average of 35% to less than 5% in weight and at the same time both insect survival rate and pest mortality rate are increased.

The coincidence mentioned was due to a filling error when a mixture of various alcohols and acetone was filed into a vessel containing a residual amount of hydrogen peroxide and an essential oil was filled in afterwards. In the mixture containing hydrogen peroxide it was found that a substance precipitated in the form of white flakes in the previously clear mixture. It can be attributed to the inventor's curiosity who tried out the mixture regarded as useless on a bee colony kept in quarantine and meant to be destroyed because it was heavily infested with mites. The colony was totally cleared of mites after application of the ‘accidental’ mixture.

At the time of application the exact mode of action was yet unknown. It is assumed that by adding the oxidising agent hydrogen peroxide a redox reaction with one of the alcohols or with acetone was initiated. The substances resulting from this reaction especially the oxide component OX was not definable by the applicant at the time of application.

A total of 24 comparative tests the representative results of which are outlined by means of the table below demonstrate this synergistic effect. Two bee colonies each were treated for a fortnight with the invented substance called NOE SS 25 and with a mixture of 33% water, 54% alcohol, 3% acetone and 10% thymol.

In the beginning each of the hives was populated with approx. 25,000 bees. The colonies were housed in two so-called ‘Herold’ or ‘Zeitler’ hives with 10 frames. The figures in the table represent the degree of mite infestation, i.e. the amount of dead mites fallen down inside the hive. The accuracy of the mite count was guaranteed by exchanging the floor panel covering once a week.

| | | |
|---|---|---|
| Substance | NOE SS 23 | Thymol 10 |

| Colonies | BV 1 | BV 2 | BV 3 | BV 4 |
|---|---|---|---|---|
| *1. | 0 | 0 | 0 | 0 |
| *2. | 0 | 0 | 0 | 0 |
| *3. | 0 | 2 | 5 | 3 |
| *4. | 0 | 0 | 8 | 9 |
| 5. | 0 | 1 | 9 | 14 |
| 6. | 0 | 0 | 15 | 17 |
| 7. | 2 | 4 | 33 | 23 |
| *8. | 0 | 2 | 35 | 37 |
| *9. | 1 | 1 | 42 | 39 |
| *10. | 1 | 0 | 47 | 58 |
| **11. | 1 | 2 | 750 | 1,050 |
| **12. | 4 | 7 | 1,050 | 1,350 |
| 13. | 6 | 3 | 19 | 14 |
| 14. | 1 | 1 | 3 | 2 |
| total | 16 | 23 | 2,016 | 2,616 |

In the first four weeks as well as in the 8^{th} to 10^{th} week 70 ml of the respective substance mixtures were sprayed into the frames. In the 11^{th} and 12^{th} week all four colonies were treated with Perizin® in a drip process as specified in the instructions of use in order to verify the mite infestation still existing.

In bee colony BC 1 and BC 2 mite infestation was continuously low at a weekly average of 0.7 mites up to the treatment with Perizin®. Mite infestation in BC 3 and BC 4 was continuously rising with a weekly average of 19.7 mites, i.e. 30 times as high. After 10 weeks a treatment with Perizin® was carried out for ascertaining information on the amount of mites in the hive still alive. In BC 1 and BC 2 an average of 7 mites was counted over two weeks. In BC 3 and BC 4 it was an average of 2,100 mites, i.e. 300 times as many.

This result shows that the mixture according to the invention does not only successfully prevent mite reproduction, but also fights all mites. The thymol mixture on the other hand does not prevent reproduction of the mites and hits only a fraction of the mites.

In addition, bee mortality is much lower with NOE SS 23 than with Thymol 10. With NOE SS 23 bee mortality was at 40 per week which is only slightly above the natural mortality rate of around 30 bees. With Thymol 10 bee mortality was at around 270 per week corresponding to 15% of all bees in the hive over the treatment period of two weeks.

Before the comparative tests the mixture according to the invention was applied on a first bee colony and results were compared with an untreated colony. These tests will be outlined in detail after this description.

The favourable effect of the pests dying while beneficial insects remain unharmed when applying the mixture according to the invention is due to fact that because of the oxide component OX a more accurate dosage of the essential oil is possible. On the other hand, it has a fatal effect on the pest because of the dosage. On which animals the mixture has a fatal effect is ultimately a matter of dosage and/or concentration of components. For smaller animals a lower concentration is sufficient by which larger animal are not affected. This effect is used for controlling parasites like mites, as mites are many times smaller than for instance bees.

A major advantage in this context is that the water solubility of the oxide component OX is changing in such a way that the essential oil or the phenol is dissolved much better and a more homogenous dosing is possible. Unlike with the conventional method the molecules of the essential oil not soluble in water are not dissolved by acetone but by the oxide component OX. As outlined below water only serves the purpose of diluting the invented mixture.

Alcohol is serving as an innoxious carrier substance. As it has a very low surface tension the pest and the animal to be treated are evenly wetted with it. Due to this homogeneous distribution the dose of the oxide component can be accurately controlled. Concentration of the essential oil can be reduced to less than 5% in weight which is sufficient to fight the pest but harmless for the beneficial insect.

Alcohol has excellent diffusion and wetting properties and is a very good solvent so that after the wetting process alcohol as well as ketone, essential oil, and oxide component are penetrating through the cell walls of the mites' nests and also through the skin or cuticle of the pest. One example of a cell wall is the capping of the bee larva, which is serving as a nest for the mite. Alcohol is also innoxious for the treated animal and has an anaesthetising effect.

Another favourable property of this mixture is that the animal to be treated is entirely wetted and as surface tension of the mixture is extremely low it easily penetrates into body openings of the animal to be treated as well as of the pest without the pest having a chance of averting this damage. The mixture penetrates for instance into spiders through the opening used for food intake. All pests present in the digestive tract of the spider are then equally wetted, the mixture penetrates them as well and they die off.

The mixture is applied either by way of evaporation or in the form of finest driblets sprayed into the nest of the animal to be treated, for instance into the bee hive. Another possibility is to apply an atomised spray or driblets directly onto the individual animal to be treated.

The mixture contains in particular a component A consisting of a primary or secondary alcohol of the general formula R1-OH, R1 being a branched or linear alkyl radical with 1 to 12, preferably 1 to 6 carbon atoms connected to the hydroxyl group, a component B of the ketone family consisting of at least one carbonyl compound of the general formula R2—CO—R2, R2 or R2′ being a branched or linear aryl or alkyl radical or hydrogen connected to the carbonyl group. R2 and R2′ can be identical or different. Component C contains at least one substance based on the aromatic hydroxyl compound benzene of the general formula C₆H₆ from the family of essential oils.

The substance is made by mixing at least one of the components A, B, and C with at least one further component D and by subsequently mixing all components, wherein component D is an oxidising agent which will react with at least one of the components A to C in a redox reaction so that the oxidising agent is reduced. Preferably components A and B are mixed with component D and subsequently component C is added.

An essential oil in the sense of the invention is a mixture consisting mainly of an aromatic hydroxyl compound of the formula R3-OH. R3 consists mainly of at least one annular aryl residue which is in turn mono- or polysubstituted. Connected to the aryl radical is at least one organic compound R3′, R3′ being an aryl, alkyl, or cycloalkyl radical. Annular aryl radicals with 6 carbon atoms are preferred. The aryl radical is branched or linear.

Synthetic and almost pure active agents of essential oils are yet in practice mixed with other substances or impurities. Natural essential oils usually contain only 70% of the chemically pure hydroxyl compound, which means that apart from the prevalent hydroxyl compound there can be hundreds of other substances and impurities.

Particularly good results can be achieved if component A contains at least 1-propanol of the molecular formula CH₃—CH₂—CH₂—OH and/or 2-propanol of the molecular formula CH₃—CH—OH—CH₃. As opposed to other alcohols propanol with its longer chains is less of a health hazard; it is volatile and makes sure the mixture is transported swiftly. Generally propanol is particularly advantageous because it mixes well with the other ingredients and can be produced at low costs.

Another advantage is that component B contains at least acetone (propanone) of the molecular formula CH₃—CO—CH₃ which further increased effectiveness of the substance. As opposed to other ketones acetone is a particularly good solubilizer. The various components of the mixture like components A and C have different densities which means that on a molecular level there will be an accumulation of some of the components. Such accumulations or uneven mixing ratios must be avoided by all means because exceedingly high or low concentrations of the individual components within the dispensed quantity can cause a loss in effectiveness of the whole mixture or the beneficial insect might be harmed. A sustained even distribution of water, alcohol, and oil in the mixture is also brought about by oxidation of the aromatic compound in the oil.

It is also an advantage that component C contains at least phenol of the general formula C₆H₅—OH and/or phenol ether of the general formula C₆H₅—O—R. Phenols and phenol ethers are contained in cloves oil (eugenol), for instance which brings about particularly good results.

The advantage of alcohol is that the body openings of a bee or a spider as well as the feeding opening of the pest are entirely wetted with the mixture. Upon wetting the mixture penetrates into the body opening, without patient or pest having a chance of defending themselves against it. The wetting process is therefore continuing inside the body so that the mixture is getting into the whole digestive tract without patient or pest having a chance of closing body openings in order to avoid penetration of the mixture.

In the case of spiders infested with round worms (nematodes) the mixture advances into the digestive tract of the spider all by itself after the food intake opening of the spider was wetted with the mixture. The nematode in turn is also wetted by the mixture which penetrates into its body, too, and produces its effect there.

Results are further improved by using cloves oil or the pure substance of eugenol as essential oil component C. Cloves oil or eugenol are known to be quite effective already in very low quantities but even a slight overdose leads to massive damage to the animal to be treated. Experts advise against using these two critical oils. Application of cloves oil and eugenol becomes safe only on the basis of the idea of the invention: achieving an even distribution of the essential oil in alcohol as carrier substance by means of oxidation and thereby allowing accurate adjustment of the dose on the basis of the easily controllable evaporation rate of alcohol and water.

Apart from cloves oil or eugenol other essential oils or their pure components (pure substances) are applied to the best advantage on their own or in combination with one or several oils or components. Other oils critical with respect to damaging the animal to be treated are garlic oil, oregano oil, thyme oil, wormwood oil, and onion oil. Less critical oils are mustard oil, aniseed oil, ylang-ylang oil, valerian oil, bergamot oil, savory oil, dill oil, staghorn sumac oil, eucalyptus oil, fennel oil, pine oil, galbanum oil, goosefoot oil, geranium oil, cloves oil, grapefruit oil, green mint oil, camomile oil, cardamom oil, pine oil, coriander oil, cumin seed oil, templin oil, lavender oil, lily oil, bay leaf oil, marguerite oil, marjory oil, tangerine oil, Melissa oil, mint oil, nutmeg oil, nimtree oil, olive oil, orange blossom oil, peppermint oil, rosemary oil, sage oil, sandalwood oil, sassafras oil, celery oil, sunflower seed oil, spike lavender oil, fir needle oil, vanillin, violet oil, juniper oil, wine yeast oil, wintergreen oil, hyssop oil, cedar oil, cinnamon oil, lemon oil, citrus oil.

Other favourable substances are chemically pure components of essential oils which are used on their own or in combination with other pure components or other oils. Chemically pure substances in the sense of the invention are e.g. acetyl eugenol, anethol, a-pinene, a-terpinene, a-thujone, benzoic acid, benzyl alcohol, bornyl acetate, camphene, caryophyllene, citral, citronellal, citronellol, coumarin, decanal, elemol, eucalyptol, eugenol, geraniol, isoeugenol, camphor, limonene, linalool, lynalyl acetate, menthol, menthone, nerolidol, octenal, cymene, phenyl acetylene, pinene, safrole, terpineol, thymol, y-terpinene, cinnamaldehyde.

The individual oils or components have anesthetising and stimulating effects in varying degrees, so that a mixture of several such substances has at least the same if not a better effect than cloves oil depending of the type of pest and the type of animal to be treated.

It is a particular advantage that component D is an inorganic oxidising agent and between 0.01 and 8.4% of component D are added to the mixture. At such a concentration no harm is done to the beneficial insect while the pest is almost entirely eliminated. It is also favourable that component C is reduced to 100% and at least one of the components A, B, or C is oxidised. Evidence can be provided that the mixture consisting of components A and B was partially oxidised before component C is added.

As an oxidising agent a 3 to 100% hydrogen peroxide solution is added as component D to the mixture. According to the invention the solution of hydrogen peroxide is added to the mix of components A and B whereupon the oxidising process is triggered.

As alternative embodiment of the invention it is contemplated that the mix of components A and B are subjected to a different oxidising process with air or oxygen and mixing in component C subsequently. For this purpose air or the gas is blown into the mixture through a lance.

For the purpose of disinfection it makes sense to add to the mixture a component E consisting of at least one aromatic alcohol of the general formula R4-OH, R4 being at least one annular aryl radical connected to the hydroxyl group. Annular aryl radicals with 6 carbon atoms are preferred. It is essential according to the invention to add component E to components A and B before component D is added to the mixture. In this way component E alone or in combination with one of the other components A or B is oxidised by component D. The additional component E shall at least make up for 0.05 to 1.9% in weight.

Aromatic alcohols just like alcohols are compounds with simple functional groups, which means that the effect of the substances as set forth above, is not influenced by the phenol. Like alcohol and ketone, phenol has an anaesthetic and a similarly diffusive and fat solving effect.

It is particularly favourable to mix in at least 2-biphenylol (2-hydroxybiphenyl or 2-phenylphenol) of the molecular formula C₆H₆—C₆H₄—OH. As opposed to other aromatic alcohols 2-biphenylol has a stabilising effect on the mixture. Stabilising means counteracting any demixing or defractionation and preventing or containing chemical reactions among the various substances. Another advantage of 2-biphenylol over other aromatic alcohols is that it is less acidic. An important advantage of 2-biphenylol over phenol is that 2-biphenylol is not carcinogenic.

The mixture contains water (H₂O) as a component F. To bring about the effect according to the invention it is necessary that the concentration of the whole mixture as well as of the individual components is high enough to destroy the pest but not so high as to harm the animal to be treated. To achieve this it makes sense to dilute the mixture with water (H₂O). According to the invention the mixture contains between 30 and 50% in weight of the additional component F before component D is added. It can be assumed that water is not involved in the redox reaction, even though it can not be totally excluded. It is advisable to add in the water first because of the material properties of hydrogen peroxide.

Furthermore it is contemplated that other carriers of animal pests be wetted with the mixture. This includes plants like trees and shrubs and their fruits, as well as grass, flowers, moss, and textiles e.g. bed linen, mattresses, clothes and underwear which can be infested with animal pests or serve as nests for such pests. In this particular embodiment, too, and due to the oxidation process, substances A, B, C, and D have the interactive effect of anesthetising the pest and to harm the central nervous system. In this type of application it is of outstanding importance that the mixture according to the invention does not represent any health hazard for human beings as they are directly in contact with the objects treated or they even consume them. In the field of grape vine protection the mixture is used for all three generations of the grape vine moth.

In order to achieve an effective composition of ingredients the mixture should contain between 50 and 70% in weight of component A, between 0.05 to 9.6% in weight of component B and between 0.05 and 3.8% in weight of component C. Component D is at first added to the mix of components A and B and if applicable E, and C is added afterwards. Concentrations of components A to F highly depend on the type of animal to be treated and of the size of the parasite. For larger animals and humans concentrations can be considerably increased.

The mixture can be applied in an advantageous way as a medicine for the treatment of invertebrates and vertebrates. On that score the use of the mixture described above is favourable for producing a medicine for the treatment of the animal body in fighting pests. The substances A to F are present in the liquid form at ambient temperatures and normal pressure and the medicine is produced in the liquid form.

At least a part of the mixture can be produced in the form of a gel or as a powder so that the mixture required for the application can be mixed e.g. with water only immediately before it is applied.

It is advantageous to use the mixture for producing a medicine for the treatment of arthropods in fighting pests. Pests like mites, lice and nematodes belong to the family of invertebrates, mites having a chitin skeleton.

An exceedingly advantageous application of the mixture is for the production of a medicine for the treatment of honeybees in fighting Varroa mites (Varroa destructor or Varroa jacobsoni). With the mixture according to the invention it was achieved that mite infestation went down by 95% compared with untreated reference colonies within a period of 6 to 8 weeks without any damage worth mentioning to the active bee colony and the brood. It was also found that the mixture can be applied in a preventive way so that mite infestation is avoided to the greatest possible extent.

In the fight against mites the mixture is applied from outside. It penetrates into a mite through the cuticle and by way of forced intake during the wetting process. It damages the central nervous system and the musculoskeletal system due to the onset of fat withdrawal as described at the beginning. The concentration of the active substances in the mixture is so low in this case that the animal to be treated like the bee is not injured as it is bigger than the mite many times over.

Subject of study was the effect of the mixture in the treatment of honeybees infested with Varroa mites.

For the test set-up 2004 two bee colonies (A and B) were chosen, which were not used for honey production because of the medium applied for subsequent evaluation of residues and also because colony B was a swarming colony. Each colony consisted of approx. 25,000 animals. Both colonies were housed each in two so-called ‘Herold’ or ‘Zeitler’ hives with 10 frames. Their infestation, colony size, brood and general state was examined and approved.

In the beginning only hive A was treated with the mixture, hive B remained untreated in order to gain reference data. Over a period of 3½ months seven treatments took place with a longer break period of more than 30 days between two treatment cycles. At the end, two treatments with Perizin® were carried out in order to check on remaining mite infestation.

For dispensing the mixture into the hive an alcohol resistant and acid-proof pressure pump sprayer made of polyethylene and equipped with a 30 cm lance was used according to specifications in manufacturer's instructions of use.

Approx. 70 ml of mixture were sprayed into each hive (i.e. into the 10 frames) at each treatment. The hive was opened and from the top the medium was finely distributed with the lance at 5 cm intervals into the spaces between the frames. First the lower than immediately the upper frame was treated.

Before the hive was covered again two minutes were allowed for aeration. The time needed for treatment of each hive was one to two minutes (without aeration).

In order to ascertain the amount of mites fallen down in the hive the floor boards of both hives (A and B) were lined entirely with two ‘diapers’ and covered with Vaseline coated paper. This was done in order to make sure that mites which might have been still alive were prevented from running away. This process was repeated after every mite count.

The colonies C to L represented reference colonies and served to ascertain the Varroa density at the location (those hives were only lined with diapers). As those were predominantly migrating colonies they were not available throughout the whole duration of the treatment, but they still represent the general situation.

Especially as many apiarists relocate their hives there is always infestation with Varroa in spite of preventive measures taken. When recording the mites found amounts of 50 animals were counted exactly. Larger amounts were partially counted and then extrapolated, as this did not play a part for the test result given this scale of infestation.

On the 30^{th} Oct. 2004 a wax and a honey sample were taken from colony A and tested for residues of the mixture. In both cases no residues were detected.

The course of the test is outlined here in note form:

6 Jun. 2004 Initial treatment. Placing of diapers in hive A and B. Checking colonies for infestation, queen, brood, and general state.

16 Jun. 2004 Check-up. No mites. 2^{nd} treatment with mixture on colony A.

27 Jun. 2004 Check-up. No mites. Treatment with mixture on colony A.

6 Jul. 2004 Check-up. No mites. Treatment with mixture on colony A.

19 Jul. 2004 Check-up. No mites. No treatment on colony A.

26 Jul. 2004 Check-up. No mites. In addition diapers in colonies C to F for ascertaining natural mite infestation.

2 Aug. 2004 Check-up. Count: A+B 2 mites each. C 18, D 8, E 6, F 6.

10 Aug. 2004 Check-up. Count: A 0, B 3, C 16, E 6, G 0. Colony G was added to the evaluation. A was treated with mixture again (2^{nd} interval).

18 Aug. 2004 Check-up. Count: A 1, B 7, C 28, D 20, E 14, G 28, F 0. A was treated again.

23 Aug. 2004 Check-up. Count: A 1, B 1, C 40, D 22, E 14, F 6, G 10. Final treatment on colony A.

30 Aug. 2004 Check-up. Count: A 1, B 2, C 58, D 50, E 40, F 6, G 8. Colonies H to L are included. In order to find out how many Varroa mites had remained in the colonies A and B were treated with Perizin®. In colonies C to L formic acid (60%) was used in an evaporation process.

6 Sep. 2004 Check-up. Count: A 4, B 60, C 1.200, D 600, E 400, F 50, G 400, H 500, I 50, J 200, K 200, L 200. Again treatment with Perizin® in colonies A and B. Treatment with formic acid in colonies C to L.

20 Sep. 2004 Check-up. Count: A 1, B 0, C 160, D 40, E 50, F 10, G 100, H 240, I 38, J 44, K 60, L 100.

Light-coloured mites i.e. in this case, young mites in the nymph state were not found in A in 2004. In B only very few could be found. In C to L so far only few were found. Later i.e. from Jun. 9, 2004 on more were detected which leads to the assumption that the mixture was effective also while the cells were capped. Only when the brood had hatched a higher amount of mites was observed because the young mites that had died off in the cells were then dragged out of the cell.

In colonies C to L some living adult mites were counted on the diapers until 30 Aug. 2004.

This test series was carried out with a mixture composition of which the exact volume parts of all components were not available at the date of application because of unreadable data material.

Apart from bees, spiders were treated with the mixture. As explained at the beginning spiders are infested by nematodes which colonise the digestive tract of the spiders as parasites. As opposed to bees the mixture must be dispensed orally to spiders. This is done through direct oral injection. The mixture is then automatically wetting the whole digestive tract of the spider and also reaches the nematodes without being decomposed or digested. The nematodes in turn are bound to take in the mixture orally for the most part but they are also exposed to the mixture on the outside of their bodies; this combined effect leads to the nematodes dying off. The spider is not harmed in the process. For such applications as well the mixture can be used as preventive care.

Moreover, the mixture according to the invention is related to producing a medicine for the treatment of the human body in fighting animal pests. The mixture is used particularly for producing a medicine for fighting pests of the family of invertebrates and is preferably used against mites or sarcoptic mites. Apart from the forms of applications described above it is contemplated to ad the mixture according to the invention as an additive to soaps, shampoos or body lotions. In this way direct skin contact is caused when the pests are colonising the skin.

What is claimed is:

1. A mixture, in particular a biocide, comprising components A, B, and C, wherein
I) component A comprises a primary or secondary alcohol or a combination thereof, of the general formula R1-OH and R1 is a branched or linear alkyl radical with 1 to 12 carbon atoms connected to the hydroxyl group and
II) component B comprises a carbonyl compound of the general formula R2-CO—R2′ and R2 or R2′ is a branched or linear aryl or alkyl radical or hydrogen connected to the carbonyl group, R2 an R2′ being identical or different and
III) component C comprises a substance on the basis of an aromatic hydroxyl compound benzene of the general formula C₆H₆ from a family of essential oils
which can be produced by way of mixing at least one or several of the components A, B, and C with one other component D and subsequently adding the remaining components to the mixture containing component D, component D being an oxidising agent which reacts with one of the components A to C in such a way that the oxidising agent is reduced.

2. The mixture according to claim 1, wherein component C comprises phenol of the general formula C₆H₅—OH or a phenyl ether of the general formula C₆H₅—O—R or a combination thereof.

3. The mixture according to claim 1 wherein component D is an inorganic oxidising agent and between 0.01 and 8.4% in weight of component D are added to the mixture.

4. The mixture according to claim 1, wherein component C is reduced by up to 100% and at least one of the components A or B or C is oxidised.

5. The mixture according to claim 1, wherein component D is at least partially formed by a 3 to 100% hydrogen peroxide solution.

6. The mixture according to claim 1, wherein the mixture contains at least between 50 and 70% in weight of component A and 0.05 and 9.6% in weight of component B and 0.05 and 3.8% in weight of component C before component D is added.

7. The mixture according to claim 1, which contains a further component E, wherein component E contains a an aromatic alcohol of the general formula R4-OH, R4 being at least one annular aryl radical connected to the hydroxyl group, the mixture contains at least between 0.05 and 1.9% in weight of the additional component E before component D is added and component E can be oxidised by component D.

8. The mixture according to claim 1, which contains a further component F, wherein component F contains H₂O or is made from H₂O and the mixture contains between 30 and 50% in weight of the additional component F before component D is added.

9. The mixture according to claim 1, wherein component A contains at least 1-propanol of the molecular formula CH₃—CH₂—CH₂—OH or 2-propanol of the molecular formula CH₃—CH—OH—CH₃, or a combination thereof.

10. The mixture according to claim 1, wherein component B comprises acetone (propanone) of the molecular formula CH₃—CO—CH₃.

11. The mixture according to claim 1, wherein component E comprises 2-biphenylol (2-hydroxy biphenyl or 2-phenyl phenol) of the molecular formula C₆H₆—C₆H₄—OH.

12. A method for forming a mixture, in particular a biocide, comprising the steps of: producing a medicine for the treatment of animal bodies in combating animal pests, comprising components A, B, and C, wherein
I) component A comprises a primary or secondary alcohol or a combination thereof, of the general formula R1-OH and R1 is a branched or linear alkyl radical with 1 to 12 carbon atoms connected to the hydroxyl group and
II) component B comprises a carbonyl compound of the general formula R2-CO—R2′ and R2 or R2′ is a branched or linear aryl or alkyl radical or hydrogen connected to the carbonyl group, R2 an R2′ being identical or different and
III) component C comprises a substance on the basis of an aromatic hydroxyl compound benzene of the general formula C₆H₆ from family of essential oils
which can be produced by way of mixing at least one or several of the components A, B, and C with one other component D and subsequently adding the remaining components to the mixture containing component D, component D being an oxidising agent which reacts with one of the components A to C in such a way that the oxidising agent is reduced.

13. A method for applying a mixture, comprising the steps of: treating one or more of an animal body and a human body to combat an animal pest with a mixture, the mixture comprising:
I) component A comprises a primary and/or or secondary alcohol or a combination thereof, of the general formula R1-OH and R1 is a branched or linear alkyl radical with 1 to 12 carbon atoms connected to the hydroxyl group and
II) component B comprises a carbonyl compound of the general formula R2 —CO—R2′ and R2 or R2′ is a branched or linear aryl or alkyl radical or hydrogen connected to the carbonyl group, R2 an R2′ being identical or different and
III) component C comprises a substance on the basis of an aromatic hydroxyl compound benzene of the general formula C₆H₆ from a family of essential oils
which can be produced by way of mixing at least one or several of the components A, B, and C with one other component D and subsequently adding the remaining components to the mixture containing component D, component D being an oxidising agent which reacts with one of the components A to C in such a way that the oxidising agent is reduced.

14. (canceled)

15. The mixture according to claim 1, wherein R1 is a branched or linear alkyl radical with 1 to 6 carbon atoms.

16. The mixture according to claim 2, wherein component D is an inorganic oxidising agent and between 0.01 and 8.4% in weight of component D are added to the mixture, wherein component C is reduced by up to 100% and at least one of the components A or B or C is oxidised, and wherein component D is at least partially formed by a 3 to 100% hydrogen peroxide solution.

17. The mixture according to claim 16, wherein the mixture contains at least between 50 and 70% in weight of component A and 0.05 and 9.6% in weight of component B and 0.05 and 3.8% in weight of component C before component D is added, which contains a further component E, wherein component E contains a an aromatic alcohol of the general formula R4-OH, R4 being at least one annular aryl radical connected to the hydroxyl group, the mixture contains at least between 0.05 and 1.9% in weight of the additional component E before component D is added and component E can be oxidised by component D, and which contains a further component F, wherein component F contains H₂O or is made from H₂O and the mixture contains between 30 and 50% in weight of the additional component F before component D is added.

18. The mixture according to claim 17, wherein component A contains at least 1-propanol of the molecular formula CH₃—CH₂—CH₂—OH or 2-propanol of the molecular formula CH₃—CH—OH—CH₃, or a combination thereof, wherein component B comprises acetone (propanone) of the molecular formula CH₃—CO—CH₃, and wherein component E comprises 2-biphenylol (2-hydroxy biphenyl or 2-phenyl phenol) of the molecular formula C₆H₆—C₆H₄—OH.

19. The mixture according to claim 1, which contains a further component E, wherein component E contains a an aromatic alcohol of the general formula R4-OH, R4 being at least one annular aryl radical connected to the hydroxyl group, the mixture contains at least between 0.05 and 1.9% in weight of the additional component E before component D is added and component E can be oxidised by component D, and which contains a further component F, wherein component F contains H₂O or is made from H₂O and the mixture contains between 30 and 50% in weight of the additional component F before component D is added.

20. The mixture according to claim 2, which contains a further component E, wherein component E contains a an aromatic alcohol of the general formula R4-OH, R4 being at least one annular aryl radical connected to the hydroxyl group, the mixture contains at least between 0.05 and 1.9% in weight of the additional component E before component D is added and component E can be oxidised by component D, and which contains a further component F, wherein component F contains H₂O or is made from H₂O and the mixture contains between 30 and 50% in weight of the additional component F before component D is added.
